(19) United States
(12) Patent Application Publication
(10) Pub. No.: US 2009/0022686 A1
(43) Pub. Date: Jan. 22, 2009

(54) PHARMACEUTICAL COMPOSITIONS FOR THE TREATMENT OF SYSTEMIC INFLAMMATORY RESPONSE SYNDROME

(75) Inventors: Uwe RIES, Biberach (DE); Wolfgang WIENEN, Biberach (DE); Uwe SCHUEHLY, Mittelbiberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 12/238,807

(22) Filed: Sep. 26, 2008

Related U.S. Application Data
(63) Continuation of application No. 10/600,055, filed on Jun. 20, 2003, now abandoned.
(60) Provisional application No. 60/400,166, filed on Aug. 1, 2002.

(30) Foreign Application Priority Data
Jun. 20, 2002 (DE) 10227668

(51) Int. Cl. A61K 38/20; A61K 31/4184; A61K 31/616; A61K 38/45; A61P 29/00; A61K 38/16; A61K 38/49; A61K 38/48

(52) U.S. Cl. 424/85.2; 514/394; 514/161; 424/94.5; 424/94.64; 514/12

(57) ABSTRACT

The present invention relates to the use of certain benzimidazoles for preparing a pharmaceutical composition for the treatment of systemic inflammatory response syndrome and particularly sepsis.

### RELATED APPLICATION

Benefit of U.S. Non-Provisional application Ser. No. 10/600,055, filed on Jun. 20, 2003, which claims benefit of U.S. Provisional Application Ser. No. 60/400,166, filed on Aug. 1, 2002 is hereby claimed, and said Applications are herein incorporated by reference.

### DESCRIPTION OF THE INVENTION

The present invention relates to the use of specific benzimidazoles of general formula (I)

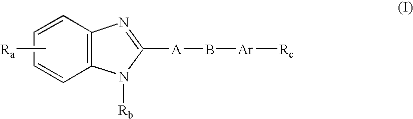

and particularly the compound of formula (II)

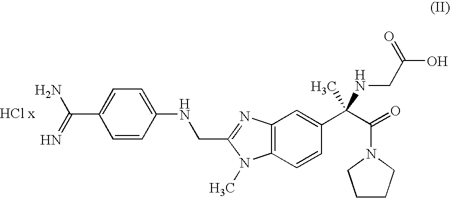

for preparing a pharmaceutical composition for the treatment of systemic inflammatory response syndrome.

“Systemic inflammatory response syndrome” (hereinafter abbreviated to “SIRS”; Bone R. C. et al.: “Definitions for Sepsis and Organ Failure and Guidelines for the Use of Innovative Therapies in Sepsis”, ACCP/SCCM Consensus Conference Committee, Chest (1992) 101: 1644) is observed in connection with insults or traumas of all kinds. The typical symptoms of a system inflammatory response occur, such as, in particular:

(a) raised or lowered body temperature of over 38° C. or below 36° C.,
(b) a heart rate of more than 90 beats per minute,
(c) rapid breathing at a rate of more than 20 breaths per minute or hyperventilation with a PaCO₂ of less than 32 mmHg,
(d) changes in the white blood picture with leukocyte numbers of more than 12,000/mm³ or less than 4,000/mm³ or the presence of more than 10% immature neutrophils.

The causes of the occurrence of SIRS are numerous. One typical cause is infections, particularly those brought about by gram-positive or gram-negative bacteria, fungi, viruses or eukaryotic single-cell organisms, as well as mixed infections with different pathogens (Balk R. A.: “Severe Sepsis and Septic Shock”, Critical Care Clinics (2000) 16: 179; Riewald M., Riess H.: “Treatment Options for Clinically Recognized Disseminated Intravascular Coagulation”, Seminars in Thrombosis and Hemostasis (1998) 24: 53). The clinical picture of sepsis or septicaemia frequently also occurs. Sepsis is defined as a general infection with symptoms arising as a result of the constant or periodic dissemination of microorganisms from a source of infection into the bloodstream. Frequently occurring sepsis pathogens are gram-negative pathogens such as e.g. Escherichia coli and other Enterobacteriaceae (Klebsiella, Proteus, Enterobacter), Pseudomonas aeruginosa, Neisseria meningitidis, Salmonella, Serratia and Bacteroides. Gram-positive pathogens include, for example, Staphylococci, Streptococci, Pneumococci, Enterococci and Clostridium perfringens. Clinical symptoms of sepsis are typically high intermittent fever, chills and significantly impaired general condition extending to confusion. As the illness progresses there may be (soft) spleen and liver enlargement as well as infectious-toxic damage to internal organs (kidneys, lungs, heart). The therapy essentially consists of treatment with antibiotics, the choice of antibiotic depending on the pathogen (often cephalosporins or penicillinase-resistant penicillins combined with an aminoglycoside). The prognosis is serious even when intensive medical treatment is administered. The mortality rate is around 50%. Older, sick or immuno-compromised patients are particularly critically affected.

Examples of non-infectious causes of SIRS are: pancreatitis, systemic and organ-limited ischaemia, trauma of various kinds (e.g. multiple bone fractures), tissue damage, large-area burns, lengthy operations, shock produced by various causes including blood loss and a condition after cardiovascular failure extending to loss of pulse and condition after resuscitation, immunomediated organ failure and inflammatory reactions induced by administering potential mediators of an inflammatory process, such as e.g. Tumour Necrosis Factor and other cytokines.

Compounds of general formula (I)

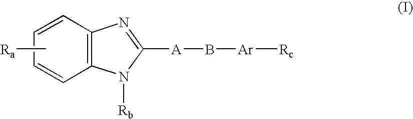

and particularly the compound (R)-2-(4-amidinophenylaminomethyl)-1-methyl-5-[1-(carboxymethylamino)-1-(pyrrolidinocarbonyl)-ethyl]-benzimidazole of formula (IIa)

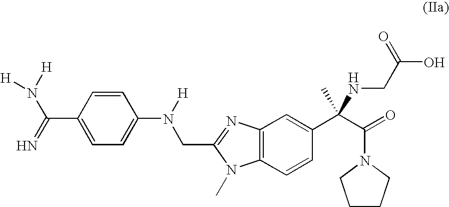

are known from WO 00/01704. Their antithrombotic activity is also known from WO 00/01704.

Surprisingly it has now been found that benzimidazoles of general formula (I)

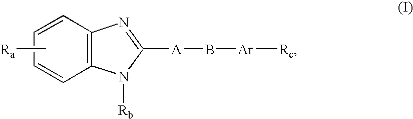

wherein

Ar denotes a phenylene or naphthylene group optionally substituted by a fluorine, chlorine or bromine atom, by a trifluoromethyl, C₁₋₃-alkyl or C₁₋₃-alkoxy group,

a thienylene, thiazolylene, pyridinylene, pyrimidinylene, pyrazinylene or pyridazinylene group optionally substituted in the carbon skeleton by a C₁₋₃-alkyl group,

A denotes a C₁₋₃-alkylene group,

B denotes an oxygen or sulphur atom, a methylene, carbonyl, sulphinyl or sulphonyl group, an imino group optionally substituted by a C₁₋₃-alkyl group wherein the alkyl moiety may be mono- or disubstituted by a carboxy group,

Rₐ denotes a R₁—CO—C₃₋₅-cycloalkyl group wherein
R₁ denotes a C₁₋₃-alkoxy, amino, C₁₋₄-alkylamino or di-(C₁₋₄-alkyl)-amino group, wherein in each case the alkyl moiety may be substituted by a carboxy group,
a 4- to 7-membered cycloalkyleneimino or cycloalkenyleneimino group which may be substituted by one or two C₁₋₃-alkyl groups, while an alkyl substituent may simultaneously be substituted by a hydroxy, C₁₋₃-alkoxy, carboxy, carboxy-C₁₋₃-alkoxy, carboxy-C₁₋₃-alkylamino, N—(C₁₋₃-alkyl)-N-(carboxy-C₁₋₃-alkyl)-amino, carboxy-C₁₋₃-alkylaminocarbonyl, N—(C₁₋₃-alkyl)-N-(carboxy-C₁₋₃-alkyl)-aminocarbonyl, carboxy-C₁₋₃-alkylaminocarbonylamino, 1-(C₁₋₃-alkyl)-3-(carboxy-C₁₋₃-alkyl)-aminocarbonylamino, 3-(C₁₋₃-alkyl)-3-(carboxy-C₁₋₃-alkyl)-aminocarbonylamino or 1,3-di-(C₁₋₃-alkyl)-3-(carboxy-C₁₋₃-alkyl)-aminocarbonylamino group,
a 4- to 7-membered cycloalkyleneimino group substituted by a hydroxy group,
a 5- to 7-membered cycloalkyleneimino group optionally substituted by a C₁₋₃-alkyl group, to which a phenyl ring is fused via two adjacent carbon atoms,
a morpholino, piperazino, N—(C₁₋₃-alkyl)-piperazino, pyrrolino, 3,4-dehydro-piperidino or pyrrol-1-yl group,

a R₂—CX—C₃₋₅-cycloalkyl group wherein
R₂ denotes a phenyl, naphthyl or monocyclic 5- or 6-membered heteroaryl group optionally substituted by a C₁₋₃-alkyl group, while the 6-membered heteroaryl group contains one, two or three nitrogen atoms and the 5-membered heteroaryl group contains an imino group optionally substituted by a C₁₋₃-alkyl group, an oxygen or sulphur atom or an imino group optionally substituted by a C₁₋₃-alkyl group and an oxygen or sulphur atom or one or two nitrogen atoms and the abovementioned alkyl substituent may be substituted by a carboxy, carboxy-C₁₋₃-alkoxy, carboxy-C₁₋₃-alkylamino or N—(C₁₋₃-alkyl)-carboxy-C₁₋₃-alkylamino group, and
X denotes an oxygen atom, a C₁₋₃-alkylimino, C₁₋₃-alkoxyimino, C₁₋₃-alkylhydrazino, di-(C₁₋₃-alkyl)-hydrazino, C₂₋₄-alkanoylhydrazino, N—(C₁₋₃-alkyl)-C₂₋₄-alkanoylhydrazino or C₁₋₃-alkylidene group each of which may be substituted by a carboxy group in the alkyl or alkanoyl moiety or in the alkyl and alkanoyl moiety,

a C₁₋₃-alkyl or C₃₋₅-cycloalkyl group substituted by an imidazole or imidazolone group, wherein
the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
the imidazolone ring may be substituted by a C₁₋₃-alkyl group, while the alkyl substituent may be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
additionally a phenyl or pyridine ring may be fused to the abovementioned imidazole and imidazolone rings via two adjacent carbon atoms,

an imidazolidin-2,4-dion-5-yl group which may be substituted by one or two C₁₋₃-alkyl groups, while simultaneously an alkyl substituent may be substituted by a carboxy group,

a C₁₋₄-alkyl group which is substituted
by a C₁₋₃-alkyl-Y₁—C₁₋₃-alkyl, HOOC—C₁₋₃-alkyl-Y₁—C₁₋₃-alkyl, tetrazolyl-C₁₋₃-alkyl-Y₂, R₃NR₄ or R₃NR₄—C₁₋₃-alkyl group and
by an isoxazolidinylcarbonyl group optionally substituted by a C₁₋₃-alkyl group, by a pyrrolinocarbonyl, 3,4-dehydro-piperidinocarbonyl, pyrrol-1-yl-carbonyl, carboxy, aminocarbonyl, C₁₋₃-alkylaminocarbonyl, di-(C₁₋₃-alkyl)-aminocarbonyl or 4- to 7-membered cycloalkyleneiminocarbonyl group, while in the abovementioned groups the cycloalkyleneimino moiety may be substituted by one or two C₁₋₃-alkyl groups and simultaneously in each case an alkyl moiety or alkyl substituent of the abovementioned C₁₋₃-alkylaminocarbonyl, di-(C₁₋₃-alkyl)-aminocarbonyl or cycloalkyleneiminocarbonyl groups may be substituted by a carboxy group, and the remaining hydrogen atoms of the C₁₋₄-alkyl group may be wholly or partly replaced by fluorine atoms, wherein
   R₃ denotes a hydrogen atom or a C₁₋₃-alkyl group optionally substituted by a carboxy group and
   R₄ denotes a hydrogen atom, a C₁₋₃-alkyl-Y₁—C₁₋₃-alkyl-Y₂, carboxy-C₁₋₃-alkyl-Y₁—C₁₋₃-alkyl-Y₂, C₁₋₃-alkyl-Y₂ or carboxy-C₁₋₃-alkyl-Y₂ group or
   R₃ and R₄ together with the nitrogen atom between them denote a 4- to 7-membered cycloalkyleneimino group optionally substituted by a carboxy, C₁₋₃-alkyl or carboxy-C₁₋₃-alkyl group, wherein
Y₁ denotes a carbon-carbon bond, an oxygen atom, a sulphenyl, sulphinyl, sulphonyl, —NH, —NH—CO or —NH—CO—NH group and
Y₂ denotes a carbon-nitrogen bond or a carbonyl, sulphonyl, imino or —NH—CO group, while the carbonyl group of the —NH—CO group is linked to the nitrogen atom of the R₃NR₄ group, and the imino groups mentioned in the definition of the groups Y₁ and Y₂ may each additionally be substituted by a C₁₋₃-alkyl or carboxy-C₁₋₃-alkyl group,

a C₁₋₃-alkyl or C₃₋₅-cycloalkyl group substituted by a R₅NR₆ group, wherein
R₅ denotes a hydrogen atom, a C₁₋₃-alkyl, C₅₋₇-cycloalkyl, phenylcarbonyl, phenylsulphonyl or pyridinyl group and
R₆ denotes a C₁₋₃-alkyl, carboxy-C₁₋₃-alkyl or carboxy-C₁₋₃-alkylcarbonyl group,

a C₁₋₃-alkyl group which is substituted by a C₂₋₄-alkanoyl or C₅₋₇-cycloalkanoyl group and by a C₁₋₃-alkyl group substituted by a chlorine, bromine or iodine atom,

R_{b} denotes a hydrogen atom or a C₁₋₃-alkyl group and

R_{c} denotes a cyano group or an amidino group optionally substituted by one or two C₁₋₃-alkyl groups, wherein
the carboxy groups mentioned in the definition of the abovementioned groups may also be replaced by a group which may be converted in vivo into a carboxy group or by a group which is negatively charged under physiological conditions or
the amino and imino groups mentioned in the definition of the abovementioned groups may also be substituted by a group which can be cleaved in vivo, while
by a group which may be converted into a carboxy group in vivo is meant a hydroxymethyl group, a carboxy group esterified with an alcohol wherein the alcoholic moiety is a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

R_{d}—CO—O—(RₑCR_{f})—OH,
wherein
R_{d} denotes a C₁₋₈-alkyl, C₅₋₇-cycloalkyl, phenyl or phenyl-C₁₋₃-alkyl group
Rₑ denotes a hydrogen atom, a C₁₋₃-alkyl, C₅₋₇-cycloalkyl or phenyl group and
R_{f} denotes a hydrogen atom or a C₁₋₃-alkyl group,
by a group which is negatively charged under physiological conditions is meant a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group
and by a group which can be cleaved from an imino or amino group in vivo is meant a hydroxy group, a benzoyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partly replaced by fluorine or chlorine atoms, a phenyl-C₁₋₆-alkoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, R_{d}—CO—O—(R_{d}CR_{f})—O—CO, C₁₋₆-alkyl-CO—NH—(R_{g}CRₕ)—O—CO or C₁₋₆-alkyl-CO—O—(R_{g}CRₕ)—(R_{g}CRₕ)—O—CO group, wherein R_{d} to R_{f} are as hereinbefore defined and
R_{g} and Rₕ, which may be identical or different, denote hydrogen atoms or C₁₋₃-alkyl groups,
   the tautomers, stereoisomers, mixtures thereof and the salts thereof, may also be used to prepare a pharmaceutical composition for the treatment or prevention of SIRS, acute cardiovascular failure, organ failure after resuscitation, acute lung failure and acute respiratory distress syndrome in adults (ARDS) and particularly sepsis.

Accordingly, the invention relates to the use of benzimidazoles of the above formula (I) wherein Ra, Rb, Rc, A, B and Ar are as hereinbefore defined, optionally in the form of the pharmaceutically acceptable acid addition salts thereof, as well as optionally in the form of the hydrates or solvates thereof, for preparing a pharmaceutical composition for the treatment of SIRS, bacteraemia and/or sepsis, including severe sepsis, acute cardiovascular failure, organ failure after resuscitation, acute lung failure and ARDS.

Preferably, benzimidazoles of general formula (Ia)

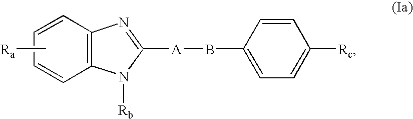

are used wherein

A denotes a C₁₋₃-alkylene group,

B denotes an oxygen or sulphur atom, a methylene, carbonyl, sulphinyl or sulphonyl group, an imino group optionally substituted by a C₁₋₃-alkyl group wherein the alkyl moiety may be mono- or disubstituted by a carboxy group,

Rₐ denotes a R₁—CO—C₃₋₅-cycloalkyl group wherein
R₁ denotes a C₁₋₃-alkoxy, amino, C₁₋₄-alkylamino or di-(C₁₋₄-alkyl)-amino group, wherein in each case the alkyl moiety may be substituted by a carboxy group,
a 4- to 7-membered cycloalkyleneimino or cycloalkenyleneimino group which may be substituted by one or two C₁₋₃-alkyl group, while an alkyl substituent may simultaneously be substituted by a hydroxy, C₁₋₃-alkoxy, carboxy, carboxy-C₁₋₃-alkoxy, carboxy-C₁₋₃-alkylamino, N—(C₁₋₃-alkyl)-N-(carboxy-C₁₋₃-alkyl)-amino, carboxy-C₁₋₃-alkylaminocarbonyl, N—(C₁₋₃-alkyl)-N-(carboxy-C₁₋₃-alkyl)-aminocarbonyl, carboxy-C₁₋₃-alkylaminocarbonylamino, 1-(C₁₋₃-alkyl)-3-(carboxy-C₁₋₃-alkyl)-aminocarbonylamino, 3-(C₁₋₃-alkyl)-3-(carboxy-C₁₋₃-alkyl)-aminocarbonylamino or 1,3-di-(C₁₋₃-alkyl)-3-(carboxy-C₁₋₃-alkyl)-aminocarbonylamino group,
a 4- to 7-membered cycloalkyleneimino group substituted by a hydroxy group,
a 5- to 7-membered cycloalkyleneimino group optionally substituted by a C₁₋₃-alkyl group, to which a phenyl ring is fused via two adjacent carbon atoms,
a morpholino, piperazino, N—(C₁₋₃-alkyl)-piperazino, pyrrolino, 3,4-dehydro-piperidino or pyrrol-1-yl group,

a R₂—CX—C₃₋₅-cycloalkyl group wherein
R₂ denotes a phenyl, naphthyl or monocyclic 5- or 6-membered heteroaryl group optionally substituted by a C₁₋₃-alkyl group, while the 6-membered heteroaryl group contains one, two or three nitrogen atoms and the 5-membered heteroaryl group contains an imino group optionally substituted by a C₁₋₃-alkyl group, an oxygen or sulphur atom or an imino group optionally substituted by a C₁₋₃-alkyl group and an oxygen or sulphur atom or one or two nitrogen atoms and the abovementioned alkyl substituent may be substituted by a carboxy, carboxy-C₁₋₃-alkoxy, carboxy-C₁₋₃-alkylamino or N—(C₁₋₃-alkyl)-carboxy-C₁₋₃-alkylamino group, and
X denotes an oxygen atom, a C₁₋₃-alkylimino, C₁₋₃-alkoxyimino, C₁₋₃-alkylhydrazino, di-(C₁₋₃-alkyl)-hydrazino, C₂₋₄-alkanoylhydrazino, N—(C₁₋₃-alkyl)-C₂₋₄-alkanoylhydrazino or C₁₋₃-alkylidene group each of which may be substituted by a carboxy group in the alkyl or alkanoyl moiety or in the alkyl and alkanoyl moiety,

a C₁₋₃-alkyl or C₃₋₅-cycloalkyl group substituted by an imidazole or imidazolone group, wherein
the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or may be substituted in the 2 or 3 position by an amino, C₂₋₄-alkanoylamino, C₁₋₃-alkylamino, N—(C₂₋₄-alkanoyl)-C₁₋₃-alkylamino or di-(C₁₋₁₃-alkyl)-amino group, and
the imidazolone ring may be substituted by a C₁₋₃-alkyl group, while the alkyl substituent may be substituted by a carboxy group or may be substituted in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
additionally a phenyl or pyridine ring may be fused to the abovementioned imidazole and imidazolone rings via two adjacent carbon atoms,

an imidazolidin-2,4-dion-5-yl group which may be substituted by one or two C₁₋₃-alkyl groups, while simultaneously an alkyl substituent may be substituted by a carboxy group,

a C₁₋₄-alkyl group which is substituted
by a C₁₋₃-alkyl-Y₁—C₁₋₃-alkyl, HOOC—C₁₋₃-alkyl-Y₁—C₁₋₃-alkyl, tetrazolyl-C₁₋₃-alkyl-Y₂, R₃NR₄ or R₃NR₄—C₁₋₃-alkyl group and
by an isoxazolidin-1-ylcarbonyl group optionally substituted by a C₁₋₃-alkyl group, by a pyrrolinocarbonyl, 2,3-dehydro-piperidinocarbonyl, pyrrol-1-yl-carbonyl, carboxy, aminocarbonyl, C₁₋₃-alkylaminocarbonyl, di-(C₁₋₃-alkyl)-aminocarbonyl or 4- to 7-membered cycloalkyleneiminocarbonyl group, while in the abovementioned groups the cycloalkyleneimino moiety may be substituted by one or two C₁₋₃-alkyl groups and simultaneously in each case an alkyl moiety or alkyl substituent of the abovementioned C₁₋₃-alkylaminocarbonyl, di-(C₁₋₃-alkyl)-aminocarbonyl or cycloalkyleneiminocarbonyl groups may be substituted by a carboxy group, and the remaining hydrogen atoms of the C₁₋₄-alkyl group may be wholly or partly replaced by fluorine atoms, wherein
   R₃ denotes a hydrogen atom or a C₁₋₃-alkyl group optionally substituted by a carboxy group and
   R₄ denotes a hydrogen atom, a C₁₋₃-alkyl-Y₁—C₁₋₃-alkyl-Y₂, carboxy-C₁₋₃-alkyl-Y₁—C₁₋₃-alkyl-Y₂, C₁₋₃-alkyl-Y₂ or carboxy-C₁₋₃-alkyl-Y₂ group or
   R₃ and R₄ together with the nitrogen atom between them denote a 4- to 7-membered cycloalkyleneimino group optionally substituted by a carboxy, C₁₋₃-alkyl or carboxy-C₁₋₃-alkyl group, wherein
Y₁ denotes a carbon-carbon bond, an oxygen atom, a sulphenyl, sulphinyl, sulphonyl, —NH, —NH—CO or —NH—CO—NH group and
Y₂ denotes a carbon-nitrogen bond or a carbonyl, sulphonyl, imino or —NH—CO group, while the carbonyl group of the —NH—CO group is linked to the nitrogen atom of the R₃NR₄ group, and the imino groups mentioned in the definition of the groups Y₁ and Y₂ may each additionally be substituted by a C₁₋₃-alkyl or carboxy-C₁₋₃-alkyl group,

a C₁₋₃-alkyl or C₃₋₅-cycloalkyl group substituted by a R₅NR₆ group, wherein
R₅ denotes a hydrogen atom, a C₁₋₃-alkyl, C₅₋₇-cycloalkyl, phenylcarbonyl, phenylsulphonyl or pyridinyl group and
R₆ denotes a C₁₋₃-alkyl, carboxy-C₁₋₃-alkyl or carboxy-C₁₋₃-alkylcarbonyl group,

a C₁₋₃-alkyl group which is substituted by a C₂₋₄-alkanoyl or C₅₋₇-cycloalkanoyl group and by a C₁₋₃-alkyl group substituted by a chlorine, bromine or iodine atom,

R_{b} denotes a hydrogen atom or a C₁₋₃-alkyl group and

R_{c} denotes a cyano group or an amidino group which may be substituted by a hydroxy group, by one or two C₁₋₃-alkyl groups, by one or two C₁₋₈-alkoxycarbonyl groups,

while the carboxy, amino and imino groups mentioned in the definition of the abovementioned groups may also be substituted by a group which can be cleaved in vivo as hereinbefore defined,

as well as the tautomers, stereoisomers and salts thereof.

Preferably, also, benzimidazoles of the above general formula Ia are used wherein

A denotes a C₁₋₃-alkylene group,

B denotes an oxygen atom, a methylene, imino or N—(C₁₋₃-alkyl)-imino group wherein the alkyl moiety may be substituted by a carboxy group,

Rₐ denotes a C₃₋₅-cycloalkyl group substituted in the 1 position by the R₁—CO group, wherein
R₁ denotes a C₁₋₃-alkoxy, amino, C₁₋₄-alkylamino or di-(C₁₋₄-alkyl)-amino group, wherein in each case the alkyl moiety may be substituted by a carboxy group,
a 4- to 7-membered cycloalkyleneimino group which may be substituted by a hydroxy group or by one or two C₁₋₃-alkyl groups, while an alkyl substituent may simultaneously be substituted by a hydroxy, C₁₋₃-alkoxy, carboxy, carboxy-C₁₋₃-alkoxy, carboxy-C₁₋₃-alkylamino, N—(C₁₋₃-alkyl)-N-(carboxy-C₁₋₃-alkyl)-amino, carboxy-C₁₋₃-alkylaminocarbonyl, N—(C₁₋₃-alkyl)-N-(carboxy-C₁₋₃-alkyl)-aminocarbonyl, carboxy-C₁₋₃-alkylaminocarbonylamino, 1-(C₁₋₃-alkyl)-3-(carboxy-C₁₋₃-alkyl)-aminocarbonylamino, 3-(C₁₋₃-alkyl)-3-(carboxy-C₁₋₃-alkyl)-aminocarbonylamino or 1,3-di-(C₁₋₃-alkyl)-3-(carboxy-C₁₋₃-alkyl)-aminocarbonylamino group,
a 5- to 7-membered cycloalkyleneimino group optionally substituted by a C₁₋₃-alkyl group, to which a phenyl ring is fused via two adjacent carbon atoms,
a morpholino, piperazino, N—(C₁₋₃-alkyl)-piperazino, pyrrolino, 3,4-Dehydro-piperidino or pyrrol-1-yl group,

a C₃₋₅-cycloalkyl group substituted in the 1 position by the R₂—CX group wherein
R₂ denotes a phenyl, naphthyl or monocyclic 5- or 6-membered heteroaryl group optionally substituted by a C₁₋₃-alkyl group, while the 6-membered heteroaryl group contains one, two or three nitrogen atoms and the 5-membered heteroaryl group contains an imino group optionally substituted by a C₁₋₃-alkyl group, an oxygen or sulphur atom or an imino group optionally substituted by a C₁₋₃-alkyl group and an oxygen or sulphur atom or one or two nitrogen atoms and the abovementioned alkyl substituent may be substituted by a carboxy, carboxy-C₁₋₃-alkoxy, carboxy-C₁₋₃-alkylamino or N—(C₁₋₃-alkyl)-carboxy-C₁₋₃-alkylamino group, and
X denotes an oxygen atom, a C₁₋₃-alkylimino, C₁₋₃-alkoxyimino or C₁₋₃-alkylidene group each of which may be substituted in the alkyl or alkoxy moiety by a carboxy group,

a C₁₋₃-alkyl group substituted in the 1 position by an imidazole or imidazolone group, wherein
the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or may be substituted in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
the imidazolone ring may be substituted by a C₁₋₃-alkyl group, while the alkyl substituent may be substituted by a carboxy group or may be substituted in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
additionally a phenyl or pyridine ring may be fused to the abovementioned imidazole and imidazolone rings via two adjacent carbon atoms,

an imidazolidin-2,4-dion-5-yl group which may be substituted by one or two C₁₋₃-alkyl groups, while simultaneously an alkyl substituent may be substituted by a carboxy group,

a C₁₋₄-alkyl group which is substituted in the 1 position
by a R₃NR₄ or R₃NR₄—C₁₋₃-alkyl group and
by a pyrrolinocarbonyl, 2,3-dehydro-piperidinocarbonyl, imidazol-1-yl-carbonyl, carboxy, aminocarbonyl, C₁₋₃-alkylaminocarbonyl, di-(C₁₋₃-alkyl)-aminocarbonyl, isoxazolidin-1-ylcarbonyl or 4- to 7-membered cycloalkyleneiminocarbonyl group, while in the abovementioned groups the cycloalkyleneimino moiety may be substituted by one or two C₁₋₃-alkyl groups and simultaneously in each case an alkyl moiety or alkyl substituent of the abovementioned C₁₋₃-alkylaminocarbonyl, di-(C₁₋₃-alkyl)-aminocarbonyl or cycloalkyleneiminocarbonyl groups may be substituted by a carboxy group, and the remaining hydrogen atoms of the C₁₋₄-alkyl group may be wholly or partly replaced by fluorine atoms, while
   R₃ denotes a hydrogen atom or a C₁₋₃-alkyl group optionally substituted by a carboxy group and
   R₄ denotes a hydrogen atom, C₁₋₃-alkyl-Y₂ or carboxy-C₁₋₃-alkyl-Y₂ group or
   R₃ and R₄ together with the nitrogen atom between them denote a 4- to 7-membered cycloalkyleneimino group optionally substituted in the 1 position by a carboxy, C₁₋₃-alkyl or carboxy-C₁₋₃-alkyl group, wherein
Y₂ denotes a carbon-nitrogen bond or a carbonyl, imino or —NH—CO group, while the carbonyl group of the —NH—CO group is linked to the nitrogen atom of the R₃NR₄ group, and the imino group occurring in the definition of the group Y₂ may additionally be substituted by a C₁₋₃-alkyl or carboxy-C₁₋₃-alkyl group,

a C₁₋₃-alkyl or C₃₋₅-cycloalkyl group substituted in the 1 position by a R₅NR₆ group, wherein
R₅ denotes a hydrogen atom, a C₁₋₃-alkyl, C₅₋₇-cycloalkyl, phenylcarbonyl, phenylsulphonyl or pyridinyl group and
R₆ denotes a C₁₋₃-alkyl, carboxy-C₁₋₃-alkyl or carboxy-C₁₋₃-alkylcarbonyl group,

a C₁₋₃-alkyl group which is substituted by C₂₋₄-alkanoyl or C₅₋₇-cycloalkanoyl group and by a C₁₋₃-alkyl group substituted by a chlorine, bromine or iodine atom,

R_{b} denotes a C₁₋₃-alkyl group and

R_{c} denotes an amidino group optionally substituted by a 2,2,2-trichloroethoxycarbonyl, C₁₋₈-alkoxycarbonyl, acetoxymethyloxycarbonyl, benzyloxycarbonyl or benzoyl group, while the benzoyl moiety may be mono- or disubstituted by fluorine, chlorine, bromine or iodine atoms or by C₁₋₃-alkyl or C₁₋₃-alkoxy groups and the substituents may be identical or different,

as well as the C₁₋₃-alkanolesters thereof and the tautomers, stereoisomers and salts thereof.

In a particularly preferred embodiment, the abovementioned pharmaceutical composition is prepared using benzimidazoles of the above general formula (Ia) wherein

A denotes a methylene group,

B denotes an oxygen atom or an imino group,

Rₐ denotes a cyclopropyl group substituted in the 1 position by the R₁—CO group wherein
R₁ denotes a pyrrolidino or piperidino group optionally substituted by a methyl or ethyl group, wherein in each case the methyl or ethyl moiety may be substituted by a carboxy, carboxy-C₁₋₃-alkoxy, carboxy-C₁₋₃-alkylamino or N—(C₁₋₃-alkyl)-carboxy-C₁₋₃-alkylamino group,

a cyclopropyl group substituted in the 1 position by the R₂—CX group wherein
R₂ denotes a phenyl, pyridyl or pyrazolyl group optionally substituted by a C₁₋₃-alkyl group, and
X denotes an oxygen atom, a C₁₋₃-alkoxyimino or C₁₋₃-alkylidene group each of which is substituted by a carboxy group in the alkyl or alkoxy moiety,
a C₁₋₂-alkyl group substituted in the 1 position by an imidazole group, wherein the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or may be substituted in the 2 or 3 position by an amino, C₂₋₄-alkanoylamino, C₁₋₃-alkyl-amino, N—(C₂₋₄-alkanoyl)-C₁₋₃-alkylamino or di-(C₁₋₃-alkyl)-amino group, while additionally a phenyl or pyridine ring may be fused to the abovementioned imidazole rings via two adjacent carbon atoms,
a C₁₋₂-alkyl group substituted in the 1 position by a benzimidazolon-1-yl group, while the imidazolone ring may be substituted by a methyl or ethyl group optionally substituted by a carboxy group,

a methyl or ethyl group which is substituted in the 1 position
by a R₃NR₄ or R₃NR₄—C₁₋₃-alkyl group and
by a di-(C₁₋₃-alkyl)-aminocarbonyl group, by an isoxazolidin-1-ylcarbonyl group, by a pyrrolidinocarbonyl or piperidinocarbonyl group optionally substituted by a C₁₋₃-alkyl group, while in the abovementioned groups in each case an alkyl moiety or alkyl substituent may be substituted by a carboxy group, while
   R₃ denotes a hydrogen atom or a C₁₋₃-alkyl group optionally substituted by a carboxy group and
   R₄ denotes a hydrogen atom, a C₁₋₃-alkyl-Y₂ or carboxy-C₁₋₃-alkyl-Y₂ group or
   R₃ and R₄ together with the nitrogen atom between them denote a 4- to 7-membered cycloalkyleneimino group optionally substituted by a carboxy group, wherein
Y₂ denotes a carbon-nitrogen bond, a carbonyl group or an imino group optionally substituted by a C₁₋₃-alkyl group,

a C₁₋₂-alkyl group substituted in the 1 position by a R₅NR₆ group wherein
R₅ denotes a pyridinyl, phenylcarbonyl or phenylsulphonyl group and
R₆ denotes a C₁₋₃-alkyl or carboxy-C₁₋₃-alkyl group,

an n-propyl group substituted in the 3 position by a chlorine atom, which is substituted in the 1 position by a cyclopentylcarbonyl group,

a cyclopropyl group substituted in the 1 position by a cyclopentylamino group which is substituted at the nitrogen atom by a carboxy-C₁₋₃-alkylcarbonyl group, R_{b} denotes a methyl group and

R_{c} denotes an amidino group optionally substituted by a C₁₋₈-alkoxycarbonyl, acetoxymethyloxycarbonyl, 2,2,2-trichloroethoxycarbonyl, benzyloxycarbonyl or benzoyl group,

or the C₁₋₃-alkanol esters, the tautomers, the stereoisomers or the salts thereof.

It is also preferable to use benzimidazoles of the above general formula (Ia) wherein

A denotes a methylene group,

B denotes an imino group,

Rₐ denotes a cyclopropyl group substituted in the 1 position by the R₁—CO group, wherein
R₁ denotes a pyrrolidino or piperidino group optionally substituted by a methyl or ethyl group, wherein in each case the methyl or ethyl moiety may be substituted by a carboxy, carboxy-C₁₋₃-alkoxy, carboxy-C₁₋₃-alkylamino or N—(C₁₋₃-alkyl)-carboxy-C₁₋₃-alkylamino group,

a cyclopropyl group substituted in the 1 position by the R₂—CX group wherein
R₂ denotes a phenyl, pyridyl or pyrazolyl group optionally substituted by a C₁₋₃-alkyl group and
X denotes an oxygen atom, a C₁₋₃-alkoxyimino or C₁₋₃-alkylidene group each of which is substituted in the alkyl or alkoxy moiety by a carboxy group,

a C₁₋₂-alkyl group substituted in the 1 position by an imidazole group wherein the imidazole ring may be substituted by 1 to 3 methyl groups or is substituted by two methyl groups and an ethyl group, while additionally one of the abovementioned methyl or ethyl substituents may simultaneously be substituted by a carboxy group,

a methyl or ethyl group which may be substituted in the 1 position
by a R₃NR₄ or R₃NR₄—CH₂ group and
by a di-(C₁₋₃-alkyl)-aminocarbonyl group, a pyrrolidinocarbonyl or piperidinocarbonyl group optionally substituted by a C₁₋₃-alkyl group, while in the abovementioned groups in each case an alkyl moiety or alkyl substituent may be substituted by a carboxy group, wherein
   R₃ denotes a hydrogen atom or a C₁₋₃-alkyl group optionally substituted by a carboxy group and
   R₄ denotes a C₁₋₃-alkyl-Y₂ or carboxy-C₁₋₃-alkyl-Y₂ group, wherein
Y₂ denotes a carbon-nitrogen bond, a carbonyl group or an imino group optionally substituted by a C₁₋₃-alkyl group,

R_{b} denotes a methyl group and

R_{c} denotes an amidino group optionally substituted by a C₁₋₈-alkoxycarbonyl, acetoxymethyloxycarbonyl, 2,2,2-trichloroethoxycarbonyl, benzyloxycarbonyl or benzoyl group,

or the C₁₋₃-alkanol esters, the tautomers, the stereoisomers or the salts thereof.

Most particularly preferred is the use of benzimidazoles of general formula (I) above and the abovementioned substituents, wherein the group Rₐ is in the 5 position, or the tautomers, the stereoisomers or the salts thereof.

Examples of preferred compounds are:
(a) 2-(4-amidinophenylaminomethyl)-1-methyl-5-[1-(pyrrolidin-1-yl-carbonyl)cyclopropyl]-benzimidazole,
(b) (E/Z)-2-(4-amidinophenylaminomethyl)-1-methyl-5-[1-[(pyridin-2-yl)-(carboxymethyloxyimino)methylene]cyclopropyl]-benzimidazole,
(c) 2-(4-amidinophenylaminomethyl)-1-methyl-5-[1-(2-carboxy-ethylamino)-1-(pyrrolidin-1-yl-carbonyl)-ethyl]-benzimidazole,
(d) 2-(4-amidinophenylaminomethyl)-1-methyl-5-[1-[2-(2-carboxyethyl)-pyrrolidin-1-yl-carbonyl]cyclopropyl]-benzimidazole,
(e) 2-(4-amidinophenylaminomethyl)-1-methyl-5-[2-(2-carboxyethyl)-4,5-dimethyl-imidazol-1-yl-methyl]-benzimidazole,
(f) 2-(4-amidinophenylaminomethyl)-1-methyl-5-[1-(carboxymethylamino)-1-(pyrrolidin-1-yl-carbonyl)-ethyl]-benzimidazole,
(g) 2-(4-amidinophenylaminomethyl)-1-methyl-5-[1-(N-methyl-carboxymethylcarbonylaminomethyl)-1-methyl-1-(pyrrolidin-1-yl-carbonyl)-ethyl]-benzimidazole,
(h) 2-(4-amidinophenylaminomethyl)-1-methyl-5-[1-(carboxymethylamino)-1-(pyrrolidinocarbonyl)-ethyl]-benzimidazole and
(i) (R)-2-(4-amidinophenylaminomethyl)-1-methyl-5-[1-(carboxymethylamino)-1-(pyrrolidinocarbonyl)-ethyl]-benzimidazole, shown in formula (IIa),
   the C₁₋₃-alkanol esters thereof, the N—(C₁₋₈-alkoxycarbonyl)-, N-benzyloxycarbonyl- and N-benzoyl-amidines thereof, the tautomers, stereoisomers and salts thereof.

Thus, the abovementioned benzimidazole compounds may be used, for example, as free bases, as zwitterions or in the form of pharmaceutically acceptable acid addition salts. Pharmaceutically acceptable acid addition salts in this context are salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, while the salts of hydrochloric acid, hydrobromic acid, sulphuric acid, phosphoric acid and acetic acid are particularly preferred. Salts of hydrochloric acid, e.g. the monohydrochloride or dihydrochloride, are most particularly preferred.

According to a particularly preferred embodiment, the abovementioned (R)-2-(4-amidinophenylaminomethyl)-1-methyl-5-[1-(carboxymethylamino)-1-(pyrrolidinocarbonyl)-ethyl]-benzimidazole is used in the form of a free base, shown in formula (IIa)

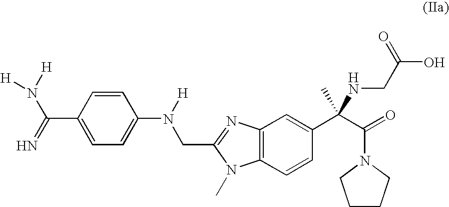

or in the form of the monohydrochloride, shown in formula (II)

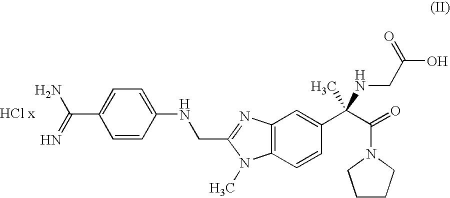

for treating the conditions mentioned above or for preparing a pharmaceutical composition for treating the conditions mentioned above. In aqueous solution, e.g. when formulated as a solution for infusion, this compound will typically be in the form of a zwitterion.

As mentioned earlier, the abovementioned compounds are known to have an antithrombotic activity, e.g. from WO 00/01704. Surprisingly, it has now been found that the abovementioned compounds and particularly compounds of formula (II) or (IIa) are suitable for treating sepsis or bacteraemia and, more generally, SIRS, acute cardiovascular failure, organ failure after resuscitation, acute lung failure and ARDS. In fact, it was known from investigations into other antithrombotic active substances known in the prior art that such active substances are not generally effective against sepsis (cf. e.g. the Kybersept Study: Warren et al., JAMA 2001; Knaub S, Keinecke H O, Juers M, Schindel F, Heinrichs H, Opal S: “High-dose antithrombin III in patients with severe sepsis—The kybersept trial” Thromb. Haemost. (2001), 6-12 Jul. 2001 (Abs P523)), i.e. it is a particular feature of the abovementioned compounds and particularly (R)-2-(4-amidinophenylaminomethyl)-1-methyl-5-[1-(carboxymethylamino)-1-(pyrrolidinocarbonyl)-ethyl]-benzimidazole.

As discussed earlier, SIRS can be subdivided into a number of subgroups. One representative subgroup consists of SIRS caused by infections, which corresponds to the clinical picture of sepsis (septicaemia; “blood poisoning”). Another subgroup would be SIRS occurring independently of infections.

Of particular clinical importance, among the non-infectious causes, is cardiovascular failure, in particular, as it occurs in a large number of patients (estimated number in the USA: up to 450,000 deaths) and with a survival rate of less than 5% it is an extremely life-threatening condition (Weisfeldt M L, JAMA 2002; 288: 3035). Even if the patient survives the first cardiac arrest the prognosis is very grave and the organ failure which persists in these patients contributes to a low survival rate even if the patient is successfully resuscitated at first. This organ failure is maintained by the combination of the activation of the clotting system during the stoppage of the cardiovascular system and the subsequent immune reaction (for the parameters of the immune reaction see Adrie C, Circulation 2002; 106: 562). Because of the combined effect of the activation of clotting and the immune reaction, the very combination of the efficacy of the compounds of formula I found according to the invention and particularly the compounds of formulae II and IIa as anticoagulants and the simultaneous reduction in the immune parameters in patients after cardiovascular stoppage is important. The compounds of formula I may be administered intravenously (as a bolus or infusion) during the resuscitation procedure, thereby increasing the likelihood of successful restoration of cardiovascular function, or intravenously (as a bolus or infusion) after cardiovascular function has been restored in order to prevent and reduce the failure of different organs. The drug can be given until the patient has recovered fully.

As SIRS progresses there may be (soft) enlargement of the spleen and liver and damage to internal organs (kidney, lung, heart). Administering the compounds of formula I according to the invention, most preferably administering compounds of formulae II and IIa, to patients at risk of developing SIRS can prevent the development of organ failure or, if SIRS is already present, may prevent further organ damage or shorten the duration of the illness.

Forms of organ failure may include: failure of heart function, which leads to a critical drop in the patient's blood pressure with further organ failure and death; kidney failure which can result in death, if left untreated, and requires the use of kidney replacement therapies (e.g. dialysis or continuous filtration) as a treatment, and acute lung failure.

Organ failure of the lung, in the form of acute lung injury (ALI) or acute respiratory distress syndrome in adults (ARDS) is particularly important, as SIRS very often leads to this outcome as a result of the combination of the activation of inflammatory reaction and clotting. Use of the compounds of formula I according to the invention, the compounds of formulae II and IIa being particularly preferred, leads to the prevention of acute lung injury in patients who may or may not be showing signs of lung injury or early forms (e.g. patients with corresponding clinical pictures [sepsis, trauma] and with no changes showing up on x-ray or with the start of lung infiltration) or for reducing the period of ventilation in patients with fully developed lung failure.

The drug may start to be administered intravenously in the form of a bolus or infusion in these clinical conditions as soon as the situation appears to be risky or it may be used as a therapy for existing signs of organ dysfunction (e.g. significantly lowered blood pressure, increasing kidney retention levels or a deterioration in the blood gases). Administration of the drug is continued as long as there is a perceived risk.

(Literature: Abraham E, Crit. Care Med 2000; 28: 232).

Accordingly the invention relates to a process for the treatment or possibly also the prevention of diseases subsumed under the heading “SIRS” or a process for preparing a pharmaceutical composition for the treatment or prevention or accompanying treatment of diseases subsumed under the heading “SIRS”, such as e.g.: SIRS caused by pathogens such as gram-negative pathogens (such as e.g. Escherichia coli, Klebsiella, Proteus, Enterobacter, Pseudomonas aeruginosa, Neisseria meningitidis, Salmonella, Serratia, Bacteroides etc.), gram-positive pathogens (such as e.g. Staphylococci, Streptococci, Pneumococci, Enterococci and Clostridium perfringens), viruses, single-cell eukaryotic parasites or fungi, SIRS with and without organ failure, septic shock, septic syndrome, SIRS caused by pancreatitis, by systemic ischaemia, by organ-limited ischaemia, by burns, by tissue damage or other trauma, SIRS occurring in connection with tumour diseases, SIRS occurring after lengthy operations, as a consequence of organ transplants or as the result of shock of various kinds, e.g. as a consequence of blood loss, following cardiovascular failure, immuno-mediated organ failure or inflammatory reactions, as well as SIRS occurring as a consequence of treatment with inflammation mediators such as for example tumour necrosis factor alpha and/or tumour necrosis factor beta and/or other cytokines, and also acute lung injury and ARDS. SIRS may be accompanied by lung damage, damage to the cardiovascular system with hypotonia, kidney failure, haematological changes, acidosis as well as multiple organ dysfunction syndrome (MODS), which can therefore also be treated using the abovementioned compounds according to the invention.

According to a preferred embodiment the invention relates to the use of (R)-2-(4-amidinophenylaminomethyl)-1-methyl-5-[1-(carboxymethylamino)-1-(pyrrolidinocarbonyl)-ethyl]-benzimidazole and particularly the monohydrochloride thereof for the treatment of sepsis, infection-related SIRS and/or SIRS as a consequence of bacteraemia, or for preparing a pharmaceutical composition for the treatment or accompanying treatment of sepsis, infection-related SIRS and/or SIRS as a consequence of bacteraemia, acute cardiovascular failure, organ failure after resuscitation, acute lung injury and ARDS.

In addition to their use as monotherapeutic agents the abovementioned benzimidazole compounds and particularly (R)-2-(4-amidinophenylaminomethyl)-1-methyl-5-[1-(carboxymethylamino)-1-(pyrrolidinocarbonyl)-ethyl]-benzimidazole may be used, according to another preferred embodiment, in conjunction with suitable additional active substances.

These might include, for example:

(a) inhibitors of platelet function such as for example acetylsalicylic acid, fibrinogen receptor antagonists (e.g. abciximab, eptifibatide, tirofiban, roxifiban), inhibitors of ADP-induced aggregation (e.g. clopidogrel, ticlopidine), P₂T receptor antagonists (e.g. cangrelor) and combined thromboxane receptor antagonists/synthetase inhibitors (e.g. terbogrel),
(b) thrombolytically active substances, such as for example alteplase, reteplase, tenecteplase, urokinase, staphylokinase and streptokinase,
(c) physiological activators and inhibitors of the clotting system and their recombinant analogues (e.g. protein C, recombinant human activated Protein C (rhAPC), tissue factor pathway inhibitor (TFPI), antithrombin),
(d) active substances conventionally used in sepsis such as e.g. substances with an antagonistic effect on endotoxins, interleukins, TNF, bradykinin, prostaglandins, cyclooxygenases, NO, PAF such as for example those which have an antagonistic effect on the associated receptors, those which interfere with the particular principle of effect and antibodies specifically directed against the substances, etc.,
(e) platelet activating factor acetylhydrolase (PAF-AH), preferably human PAF-AH (described e.g. in Tjoelker L W, Stafforini D M: “Platelet-activating factor acetylhydrolases in health and disease” Biochim Biophys Acta (2000) 1488:102-123, WO 95/09921 and WO 95/00649) as well as PAF-AH derivatives such as, in particular, shortened PAF-AH proteins as described for example in WO 99/09147,
(f) conventional therapeutic agents which are used to inhibit inflammation, such as e.g. base therapeutics for rheumatic diseases such as chloroquine, gold preparations, D-penicillamine, methotrexate, chlorambucil, cyclophosphamide, corticosteroids in all forms, cyclosporin, tacrolimus, sirolimus, azathioprin, mycophenolate mofetil, etc., and
(g) drugs which are conventionally used for treating sepsis, such as e.g. antibiotics, substances acting on the circulation such as catecholamines, etc.

The invention therefore also includes combinations of the abovementioned benzimidazole compounds and particularly (R)-2-(4-amidinophenylaminomethyl)-1-methyl-5-[1-(carboxymethylamino)-1-(pyrrolidinocarbonyl)-ethyl]-benzimidazole, optionally in the form of the pharmaceutically acceptable acid addition salts as well as optionally in the form of the hydrates or solvates thereof, with one or more of the substances listed under (a) to (g) above. The combination of (R)-2-(4-amidinophenylaminomethyl)-1-methyl-5-[1-(carboxymethylamino)-1-(pyrrolidinocarbonyl)-ethyl]-benzimidazole with a PAF-acetylhydrolase and particularly one of the PAF-AH-derivatives described in WO 99/09147 such as e.g. rPH.2 or rPH.9 mentioned therein (page 10 ff. of WO 99/09147) are found to be particularly effective. The combination of (R)-2-(4-amidinophenylaminomethyl)-1-methyl-5-[1-(carboxymethylamino)-1-(pyrrolidinocarbonyl)-ethyl]-benzimidazole with an anti-human TNFalpha-antibody, such as e.g. afelimomab (INN), is also particularly advantageous.

The abovementioned benzimidazole active substances or combinations of active substances are administered in the usual way, preferably parenterally and more preferably by intravenous route (i.v.), optionally also subcutaneously. They may be administered parenterally for example by i.v. infusion which may also in certain circumstances be given over a longer period (hours or days) (continuous long-term infusion) depending on the clinical picture. Doses for i.v. administration may be, for example, in the range from 0.05 to 2000 mg/24 h. The optimum therapeutic dose depends on the indication and the formulation used and can be determined experimentally in a manner known to those skilled in the art. The proposed dosage range for the active substance (R)-2-(4-amidinophenylaminomethyl)-1-methyl-5-[1-(carboxymethylamino)-1-(pyrrolidinocarbonyl)-ethyl]-benzimidazole, for example, is the range from 0.0001 mg/kg/h to 1 mg/kg/h, preferably from 0.001 mg/kg/h to 0.5 mg/kg/h and more preferably from 0.01 mg/kg/h to 0.3 mg/kg/h. The skilled man will naturally see that it may be necessary to deviate from the quantities specified, depending on the patient's body weight or the method of administration, the individual response to the drug, the nature of the formulation and the time or period of time when it is administered. Thus, in some cases, it may be sufficient to use less than the minimum amount specified, whereas in other cases the dosage may have to exceed the upper limit specified. When administering larger amounts it may be advisable to spread them out over the day in a number of single doses.

The skilled man will be aware of methods by which to formulate the benzimidazole active substances and combinations of active substances mentioned above for particular applications (Gennaro, Alfonso R.: Remington's Pharmaceutical Sciences. Easton Mack). Solutions for injection and infusion are prepared in the usual way, e.g. with the addition of buffers, isotonic agents, preservatives such as p-hydroxybenzoates, or stabilisers such as alkali metal salts of ethylenediaminetetraacetic acid, optionally using emulsifiers and/or dispersants, while if water is used as diluent, for example, organic solvents may optionally be added as solubilisers or auxiliary solvents, and the finished solutions transferred into injection phials or ampoules or infusion bottles.

The Examples that follow illustrate the invention without restricting its scope.

### EXAMPLES

### Example 1

### Preparation of (R)-2-(4-amidinophenylaminomethyl)-1-methyl-5-[1-(carboxymethylamino)-1-(pyrrolidinocarbonyl)-ethyl]-benzimidazole-hydrochloride

### a.) ethyl 2-amino-2-(4-chloro-3-nitro-phenyl)-propionate

A mixture of 28 g (0.11 mol) of 2-amino-2-(4-chloro-3-nitro-phenyl)-propionic acid in 200 ml of 5.6N ethanolic hydrochloric acid is refluxed for 36 hours. After the solvent has been evaporated off the residue is suspended in 300 ml of ethyl acetate and combined with 300 ml of saturated sodium hydrogen carbonate solution. The organic phase is washed twice with saturated sodium hydrogen carbonate solution and once with water, dried over sodium sulphate and concentrated by evaporation.

Yield: 21.1 g (68% of theory) light brown oil.

### b.) ethyl (R)-(+)-2-amino-2-(4-chloro-3-nitro-phenyl)-propionate

17.33 g (63.6 mmol) of ethyl 2-amino-2-(4-chloro-3-nitro-phenyl)-propionate are dissolved in 247 ml isopropanol and 207 ml of methanol and combined with 9.54 g (63.6 mmol) of L-(+)-tartaric acid. The reaction mixture is heated to 100° C., whereupon a clear solution is formed. The solution is cooled to 27° C. within 3 hours, the precipitate formed is suction filtered, washed with ethanol and dried. Then the solid formed (21.5 g) is suspended in 400 ml of ethyl acetate and combined with 400 ml of saturated sodium hydrogen carbonate solution. After extraction and phase separation the organic phase is washed with water, dried and concentrated by evaporation.

Yield: 7.68 g (44.4% of theory) of light yellow oil,

[α]₂₀=+4.380 (ethyl acetate)

HPLC analysis: ee value>98.6%

### c.) (R)-(−)-2-amino-2-(4-chloro-3-nitro-phenyl)-propionic acid

150 mg of ethyl (R)-(+)-2-amino-2-(4-chloro-3-nitro-phenyl)-propionate and 2.5 ml of 2N sodium hydroxide solution are stirred into 10 ml of tetrahydrofuran for 5 hours at ambient temperature. The tetrahydrofuran is distilled off and the residue is adjusted to pH 5 with hydrochloric acid. The crystalline product is suction filtered, washed with water and dried.

Yield: 63% of theory,

[α]²⁰=−59.60 (methanol/water 1:1)

### d.) (R)-2-tert.butyloxycarbonylamino-2-(4-chloro-3-nitro-phenyl)-propionic acid

5.7 g of (R)-(−)-2-amino-2-(4-chloro-3-nitro-phenyl)-propionic acid are dissolved in 50 ml of dioxane and after the addition of 5.5 ml (39.1 mmol) of triethylamine and 4.8 g of pyrocarbonic acid-di-tert.butyldicarbonate the mixture is stirred for 18 hours at ambient temperature. Then it is diluted with 0.5 M potassium hydrogen sulphate solution and extracted with ethyl acetate. The combined organic extracts are dried and concentrated by evaporation.

Yield: 100% of theory.

### e.) (R)-2-tert.butyloxycarbonylamino-2-(4-methylamino-3-nitro-phenyl)-propionic acid

20.0 g of (R)-2-tert.butyloxycarbonylamino-2-(4-chloro-3-nitro-phenyl)-propionic acid and 100 ml methylamine solution (40% in H₂O) are heated to 80° C. in a pressure vessel for five hours. The contents are evaporated to dryness, dissolved in water and acidified with glacial acetic acid. The product precipitated is suction filtered, washed with water and dried.

Yield: 69% of theory.

### f.) (R)-2-(4-methylamino-3-nitro-phenyl)-2-tert.butyloxycarbonylamino-1-pyrrolidino-propanone

12.3 g of (R)-2-tert.butyloxycarbonylamino-2-(4-methylamino-3-nitro-phenyl)-propionic acid (64.8 mmol) are dissolved in 90 ml of tetrahydrofuran and combined with 12.6 g (78 mmol) of carbonyldiimidazole. After 30 minutes at ambient temperature 10.9 ml (130 mmol) of pyrrolidine are added. After a further 12 hours at ambient temperature the reaction solution is combined with 800 ml of water. The solid formed is filtered off, washed with water and dried. Yield: 48% of theory.

### g.) (R)-2-(4-methylamino-3-amino-phenyl)-2-tert.butyloxycarbonylamino-1-pyrrolidino-propanone

3.1 g of (R)-2-(4-methylamino-3-nitro-phenyl)-2-tert.butyloxycarbonylamino-1-pyrrolidino-propanone (7.9 mmol) are dissolved in 30 ml of methanol and hydrogenated for 2 hours at ambient temperature with the addition of hydrogen/10% palladium on activated charcoal. The catalyst is filtered off and the solvent is distilled off. The residue is dissolved in 50 ml of methyl-tert.butylether at 45° C. The solid formed after 12 hours at 5° C. is suction filtered and dried.

Yield: 87% of theory.

### h.) (R)-2-(4-cyanophenylaminomethyl)-1-methyl-5-[1-(N-tert.butyloxycarbonylamino)-1-(pyrrolidinocarbonyl)-ethyl]-benzimidazole

11.2 g (63 mmol) of 4-cyanophenylglycine and 10.95 g (67.5 mmol) of carbonyldiimidazole are stirred in 320 ml of tetrahydrofuran at ambient temperature for 2 hours. After the addition of 23 g (60 mmol) of (R)-2-(4-methylamino-3-amino-phenyl)-2-tert.butyloxycarbonylamino-1-pyrrolidino-propanone the reaction mixture is refluxed for 2 hours. The solvent is distilled off, the residue is taken up in 320 ml glacial acetic acid and refluxed for 1 hour. The solid formed after the addition of 500 ml ice water is filtered off, washed and dried.

Yield: 96% of theory.

### i.) (R)-2-(4-cyanophenylaminomethyl)-1-methyl-5-[1-amino-1-(pyrrolidinocarbonyl)-ethyl]-benzimidazole

1.3 g of (R)-2-(4-cyanophenylaminomethyl)-1-methyl-5-[1-(N-tert.butyloxycarbonylamino)-1-(pyrrolidinocarbonyl)-ethyl]-benzimidazole are dissolved in 20 ml dioxane and after the addition of 6N hydrochloric acid stirred for two hours at ambient temperature. Ice is added to the solution, which is made alkaline with ammonia and extracted with ethyl acetate. The combined organic extracts are dried and concentrated by evaporation.

Yield: 76% of theory.

### k.) (R)-2-(4-cyanophenylaminomethyl)-1-methyl-5-[1-(ethoxycarbonylmethylamino)-1-(pyrrolidinocarbonyl)-ethyl]-benzimidazole

1.2 g of (R)-2-(4-cyanophenylaminomethyl)-1-methyl-5-[1-amino-1-(pyrrolidino-carbonyl)-ethyl]-benzimidazole are dissolved in 20 ml acetone and after the addition of 0.39 ml ethyl iodoacetate and 0.56 g of potassium carbonate the mixture is refluxed for 3 hours. The reaction mixture is filtered and concentrated by evaporation, the residue is chromatographed on silica gel, eluting with methylene chloride/ethanol (20:1 and 4:1).

Yield: 75% of theory.

### l.) (R)-2-(4-amidinophenylaminomethyl)-1-methyl-5-[1-(ethoxycarbonylmethylamino)-1-(pyrrolidinocarbonyl)-ethyl]-benzimidazole-hydrochloride

1.0 g of (R)-2-(4-cyanophenylaminomethyl)-1-methyl-5-[1-(ethoxycarbonylmethylamino)-1-(pyrrolidinocarbonyl)-ethyl]-benzimidazole are dissolved in 50 ml of saturated ethanolic hydrochloric acid and stirred for 5 hours at ambient temperature. The solvent is distilled off, the residue is dissolved in 50 ml of absolute ethanol and combined with 2.3 g (25 mmol) of ammonium carbonate. After 60 hours at ambient temperature the mixture is evaporated to dryness. The residue is chromatographed on silica gel, eluting with methylene chloride/methanol (7:1).

Yield: 95% of theory.

### m.) (R)-2-(4-amidinophenylaminomethyl)-1-methyl-5-[1-(carboxymethylamino)-1-(pyrrolidinocarbonyl)-ethyl]-benzimidazole-hydrochloride

150 mg of (R)-2-(4-amidinophenylaminomethyl)-1-methyl-5-[1-(ethoxycarbonylmethylamino)-1-(pyrrolidinocarbonyl)-ethyl]-benzimidazole and 2.5 ml of 2N sodium hydroxide solution are stirred in 10 ml of ethanol for 5 hours at ambient temperature. The alcohol is distilled off and the residue is adjusted to pH 5 with hydrochloric acid. The crystalline product is suction filtered, washed with water and dried.

Yield: 100% of theory,

C₂₅H₃₁N₇O₃×2HCl (477.57/550.5)

Mass spectrum: (M+H)⁺=478

(M−H+HCl)⁻=512/514 (Cl)

(M−H+2HCl)⁻=448/550/552 (Cl₂)

### Example 2

### Animal Model for Systemic Inflammatory Response Syndrome (SIRS): Intravenous Lipopolysaccharide Stimulation (Modified According to Isobe et al. Circulation 2001; 104: 1171-1175)

### Method 1

Rats (male, approx. 300 g CrlGlxBrlHan:Wi) were anaesthetised with pentobarbital (60 mg/kg i.p.). To maintain the anaesthetic a pentobarbital drip was set up (22.5 mg/kg/h i.p.). The carotid artery was cannulated for taking blood samples, the left jugular vein was cannulated for administration of the substance and the right jugular vein was cannulated for administering the LPS. The administration of (R)-2-(4-)-1-methyl-5-[1-(carboxymethylamino)-1-(pyrrolidinocarbonyl)-ethyl]-benzimidazole (0.1 mg/kg/h) was started at time t=−60 min as a continuous drip and maintained until the end of the experiment. One hour after the start of the infusion of the substance, a single bolus of LPS was administered (lipopolysaccharide of E. coli serotype 0127:B8 Sigma L-3129 5 mg/kg i.v. bolus) at time t=0 min. In order to determine the coagulation and inflammation parameters, blood was taken via the arterial cannulae at times a) before the infusion of placebo or (R)-2-(4-amidinophenylaminomethyl)-1-methyl-5-[1-(carboxymethylamino)-1-(pyrrolidinocarbonyl)-ethyl]-benzimidazole (−60 minutes), b) before administration of the LPS bolus (0 minutes) and c) 240 minutes after the administration of LPS. From the blood samples the TAT complex (thrombin-antithrombin) and interleukin-6 (IL-6) were determined by ELISA as parameters of a systemic inflammatory reaction.

### Results

As can be seen from Table 1, under control conditions there was no change in the measured parameters throughout the observation period. Stimulation with LPS on the other hand induced a systemic inflammatory reaction, as shown by the significant rise in the plasma levels of the TAT complex by a factor of 8.6 and the approximately 400-fold increase in the IL-6 at the end of the test period (Table 2). As can be seen from Table 3, the treatment with (R)-2-(4-amidinophenylaminomethyl)-1-methyl-5-[1-(carboxymethylamino)-1-(pyrrolidinocarbonyl)-ethyl]-benzimidazole inhibited the activation of the coagulation cascade: The plasma levels of the TAT complex were significantly lower than in the untreated LPS animals (p<0.02) and were within the control range. Surprisingly, the treatment with (R)-2-(4-amidinophenylaminomethyl)-1-methyl-5-[1-(carboxymethylamino)-1-(pyrrolidinocarbonyl)-ethyl]-benzimidazole also resulted in a significant (p<0.02) reduction in the plasma levels of IL-6 (Table 3).

TABLE 1
Controls without LPS

| Parameter | −60 min | 0 min | +240 min |
|---|---|---|---|
| TAT (ng/ml) | 4.163 +/− 1.45 | 6.45 +/− 2.78 | 5.71 +/− 1.48 |
| IL-6 (ng/ml) | 0.053 +/− 0.011 | 0.065 +/− 0.019 | 0.064 +/− 0.018 |

TABLE 2
Controls with LPS

| Parameter | −60 min | 0 min | +240 min |
|---|---|---|---|
| TAT (ng/ml) | 1.43 +/− 0.40 | 4.13 +/− 0.89 | 35.69 +/− 5.54 |
| IL-6 (ng/ml) | 0.117 +/− 0.070 | 0.158 +/− 0.067 | 62.884 +/− 5.438 |

TABLE 3
LPS + active substance (100 μg/kg/h)

| Parameter | −60 min | 0 min | +240 min | p value vs LPS |
|---|---|---|---|---|
| TAT (ng/ml) | 2.33 +/− 0.89 | 2.21 +/− 0.79 | 6.52 +/− 1.33 * | 0.021 |
| IL-6 (ng/ml) | 0.139 +/− 0.086 | 0.143 +/− 0.083 | 43.454 +/− 2.975 * | 0.022 |

Active substance = (R)-2-(4-amidinophenylaminomethyl)-1-methyl-5-[1-(carboxymethylamino)-1-(pyrrolidinocarbonyl)-ethyl]-benzimidazole (base)

### Example 3

### Animal Model for Systemic Inflammatory Response Syndrome (SIRS): Intravenous Lipopolysaccharide Stimulation (Modified According to Aoki et al. Drug Res. 50: 809; 2000 and Yamazaki et al. Blood Coag. Fibrinol. 10: 321; 1999)

### Method 2

Rats (male, approx. 300 g CrlGlxBrlHan:Wi) were anaesthetised with pentobarbital (60 mg/kg i.p.). To maintain the anaesthetic a pentobarbital drip was set up (22.5 mg/kg/h i.p.). The left jugular vein was cannulated for administration of the substance and the right jugular vein was cannulated for administering the LPS. LPS was given as a continuous infusion for 4 hours (lipopolysaccharide of E. coli serotype 0127:B8 Sigma L-3129 7.5 mg/kg/h). (R)-2-(4-amidinophenylaminomethyl)-1-methyl-5-[1-(carboxymethylamino)-1-(pyrrolidinocarbonyl)-ethyl]-benzimidazole (0.1 mg/kg/h) was administered as a continuous infusion (100 μg/kg/h) one hour after the start of the LPS infusion with a total duration of 3 hours. In order to determine the inflammation parameters, blood was taken from the abdominal artery after a total test period of four hours. From the blood samples the TAT complex (thrombin-antithrombin), interleukin-1β (IL-1β), interleukin-6 (IL-6) and the tumour necrosis factor alpha (TNF-alpha) were determined by ELISA as parameters of a systemic inflammatory reaction. Alanine-aminotransferase (ALT) and creatinin served as enzyme parameters for organ damage to the liver and kidneys.

TABLE 4

| | Controls | LPS-treated | LPS + BIBT 986 |
|---|---|---|---|
| | (Phys. NaCl) | 7.5 mg/kg/h × 4 h | 100 μg/kg/h |
| IL-1β | 17.1 ± 8.9* | 8947 ± 1501§ | 4036 ± 1000*§ |
| [pg/ml] | (10) | (8) | (8) |
| IL-6 | 31.6 ± 8.4* | 209904 ± 30076§ | 89579 ± 10069*§ |
| [pg/ml] | (10) | (7) | (8) |
| TNF-alpha | 3.2 ± 2.4* | 4241 ± 1045§ | 1003 ± 292* |
| [pg/ml] | (10) | (8) | (8) |
| ALT | 36.4 ± 7.7* | 206.9 ± 82.8§ | 44.2 ± 7.1* |
| [U/ml] | (10) | (7) | (8) |
| creatinin | 31.1 ± 3.0* | 57 ± 4.1§ | 40.1 ± 1.8* |
| [μmol/L] | (10) | (8) | (8) |

mean values ± standard deviations; number of animals shown in brackets.
*p < 0.05 vs. LPS treated animals;
§p < 0.05 vs. controls (Tukey Kramer Test)

### Results

As shown in Table 4, the continuous infusion of LPS induces a significant increase in the indicators of a systemic inflammatory process (TAT complex, IL-1β, IL-6 and TNF alpha) and markers for organ damage (ALT and creatinin). Treatment with (R)-2-(4-amidinophenylaminomethyl)-1-methyl-5-[1-(carboxymethylamino)-1-(pyrrolidinocarbonyl)-ethyl]-benzimidazole surprisingly led to a significant lowering of the TAT complex and to a significant (p<0.02) lowering of the plasma levels of IL-1β, IL-6 and TNF alpha. Moreover, in animals treated with (R)-2-(4-amidinophenylaminomethyl)-1-methyl-5-[1-(carboxymethylamino)-1-(pyrrolidinocarbonyl)-ethyl]-benzimidazole, ALT and creatinin were not significantly different from untreated control animals.

The results shown lead one to conclude that treatment with the combined factor Xa/thrombin inhibitor (R)-2-(4-amidinophenylaminomethyl)-1-methyl-5-[1-(carboxymethylamino)-1-(pyrrolidinocarbonyl)-ethyl]-benzimidazole constitutes an effective therapy for treating a systemic inflammatory response/SIRS and particularly Llpopolysaccharide-induced systemic inflammatory response and sepsis (including severe sepsis).

### Example 4

### Pharmaceutical Formulations

### a.) Dry Ampoule Containing 75 Mg of Active Substance Per 10 Ml

### Composition:

| | | |
|---|---|---|
| active substance | 75.0 | mg |
| mannitol | 50.0 | mg |
| water for injections | ad 10.0 | ml |

### Method:

Active substance and mannitol are dissolved in water. After filling the solution is freeze-dried. The solution ready for use is made up with water for injections.

### b.) Dry Ampoule Containing 35 Mg Active Substance Per 2 Ml

### Composition:

| | | |
|---|---|---|
| active substance | 35.0 | mg |
| mannitol | 100.0 | mg |
| water for injections | ad 2.0 | ml |

### Method:

Active substance and mannitol are dissolved in water. After filling the solution is freeze-dried. The solution ready for use is made up with water for injections.

### c.) Ampoule Solution

| | | |
|---|---|---|
| active substance | 2 | mg |
| sodium chloride | 9 | mg |
| water for inj. | 5 | ml |

### Example 5

### Combination of (R)-2-(4-amidinophenylaminomethyl)-1-methyl-5-[1-(carboxymethylamino)-1-(pyrrolidinocarbonyl)-ethyl]-benzimidazole with a PAF-AH

A dry ampoule according to Example 4a) or 4b) containing (R)-2-(4-amidinophenyl-aminomethyl)-1-methyl-5-[1-(carboxymethylamino)-1-(pyrrolidinocarbonyl)-ethyl]-benzimidazole monohydrochloride as active substance is placed together with a vial containing a PAF-AH lyophilisate in a combined package. To prepare a solution for infusion the dry substances are dissolved in water for injections and given to the patient by infusion together or sequentially.

What is claimed is:

1. A method of treating or preventing diseases selected from the group consisting of systemic inflammatory response syndrome (SIRS), sepsis and bacteranemia which comprises administering to a patient in need thereof a therapeutically effective amount of a benzamidazole pharmaceutical composition of general formula (I) 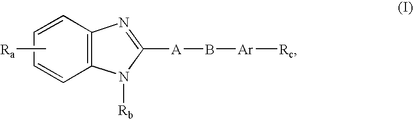
wherein
Ar denotes a phenylene or naphthylene group optionally substituted by a fluorine, chlorine or bromine atom, by a trifluoromethyl, C₁₋₃-alkyl or C₁₋₃-alkoxy group,
a thienylene, thiazolylene, pyridinylene, pyrimidinylene, pyrazinylene or pyridazinylene group optionally substituted in the carbon skeleton by a C₁₋₃-alkyl group,
A denotes a C₁₋₃-alkylene group,
B denotes an oxygen or sulphur atom, a methylene, carbonyl, sulphinyl or sulphonyl group, an imino group optionally substituted by a C₁₋₃-alkyl group wherein the alkyl moiety may be mono- or disubstituted by a carboxy group,
Rₐ denotes a R₁—CO—C₃₋₅-cycloalkyl group wherein
R₁ denotes a C₁₋₃-alkoxy, amino, C₁₋₄-alkylamino or di-(C₁₋₄-alkyl)-amino group, wherein in each case the alkyl moiety may be substituted by a carboxy group,
a 4- to 7-membered cycloalkyleneimino or cycloalkenyleneimino group which may be substituted by one or two C₁₋₃-alkyl groups, while an alkyl substituent may simultaneously be substituted by a hydroxy, C₁₋₃-alkoxy, carboxy, carboxy-C₁₋₃-alkoxy, carboxy-C₁₋₃-alkylamino, N—(C₁₋₃-alkyl)-N-(carboxy-C₁₋₃-alkyl)-amino, carboxy-C₁₋₃-alkylaminocarbonyl, N—(C₁₋₃-alkyl)-N-(carboxy-C₁₋₃-alkyl)-aminocarbonyl, carboxy-C₁₋₃-alkylaminocarbonylamino, 1-(C₁₋₃-alkyl)-3-(carboxy-C₁₋₃-alkyl)-aminocarbonylamino, 3-(C₁₋₃-alkyl)-3-(carboxy-C₁₋₃-alkyl)-aminocarbonylamino or 1,3-di-(C₁₋₃-alkyl)-3-(carboxy-C₁₋₃-alkyl)-aminocarbonylamino group,
a 4- to 7-membered cycloalkyleneimino group substituted by a hydroxy group,
a 5- to 7-membered cycloalkyleneimino group optionally substituted by a C₁₋₃-alkyl group, to which a phenyl ring is fused via two adjacent carbon atoms,
a morpholino, piperazino, N—(C₁₋₃-alkyl)-piperazino, pyrrolino, 3,4-dehydro-piperidino or pyrrol-1-yl group,
a R₂—CX—C₃₋₅-cycloalkyl group wherein
R₂ denotes a phenyl, naphthyl or monocyclic 5- or 6-membered heteroaryl group optionally substituted by a C₁₋₃-alkyl group, while the 6-membered heteroaryl group contains one, two or three nitrogen atoms and the 5-membered heteroaryl group contains an imino group optionally substituted by a C₁₋₃-alkyl group, an oxygen or sulphur atom or an imino group optionally substituted by a C₁₋₃-alkyl group and an oxygen or sulphur atom or one or two nitrogen atoms and the abovementioned alkyl substituent may be substituted by a carboxy, carboxy-C₁₋₃-alkoxy, carboxy-C₁₋₃-alkylamino or N—(C₁₋₃-alkyl)-carboxy-C₁₋₃-alkylamino group, and
X denotes an oxygen atom, a C₁₋₃-alkylimino, C₁₋₃-alkoxyimino, C₁₋₃-alkylhydrazino, di-(C₁₋₃-alkyl)-hydrazino, C₂₋₄-alkanoylhydrazino, N—(C₁₋₃-alkyl)-C₂₋₄-alkanoylhydrazino or C₁₋₃-alkylidene group each of which may be substituted by a carboxy group in the alkyl or alkanoyl moiety or in the alkyl and alkanoyl moiety,
a C₁₋₃-alkyl or C₃₋₅-cycloalkyl group substituted by an imidazole or imidazolone group, wherein
the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
the imidazolone ring may be substituted by a C₁₋₃-alkyl group, while the alkyl substituent may be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
additionally a phenyl or pyridine ring may be fused to the abovementioned imidazole and imidazolone rings via two adjacent carbon atoms,
an imidazolidin-2,4-dion-5-yl group which may be substituted by one or two C₁₋₃-alkyl groups, while simultaneously an alkyl substituent may be substituted by a carboxy group,
a C₁₋₄-alkyl group which is substituted
by a C₁₋₃-alkyl-Y₁—C₁₋₃-alkyl, HOOC—C₁₋₃-alkyl-Y₁—C₁₋₃-alkyl, tetrazolyl-C₁₋₃-alkyl-Y₂, R₃NR₄ or R₃NR₄—C₁₋₃-alkyl group and
by an isoxazolidinylcarbonyl group optionally substituted by a C₁₋₃-alkyl group, by a pyrrolinocarbonyl, 3,4-dehydro-piperidinocarbonyl, pyrrol-1-yl-carbonyl, carboxy, aminocarbonyl, C₁₋₃-alkylaminocarbonyl, di-(C₁₋₃-alkyl)-aminocarbonyl or 4- to 7-membered cycloalkyleneiminocarbonyl group, while in the abovementioned groups the cycloalkyleneimino moiety may be substituted by one or two C₁₋₃-alkyl groups and simultaneously in each case an alkyl moiety or alkyl substituent of the abovementioned C₁₋₃-alkylaminocarbonyl, di-(C₁₋₃-alkyl)-aminocarbonyl or cycloalkyleneiminocarbonyl groups may be substituted by a carboxy group, and the remaining hydrogen atoms of the C₁₋₄-alkyl group may be wholly or partly replaced by fluorine atoms, wherein
R₃ denotes a hydrogen atom or a C₁₋₃-alkyl group optionally substituted by a carboxy group and
R₄ denotes a hydrogen atom, a C₁₋₃-alkyl-Y₁—C₁₋₃-alkyl-Y₂, carboxy-C₁₋₃-alkyl-Y₁—C₁₋₃-alkyl-Y₂, C₁₋₃-alkyl-Y₂ or carboxy-C₁₋₃-alkyl-Y₂ group or
R₃ and R₄ together with the nitrogen atom between them denote a 4- to 7-membered cycloalkyleneimino group optionally substituted by a carboxy, C₁₋₃-alkyl or carboxy-C₁₋₃-alkyl group, wherein
Y₁ denotes a carbon-carbon bond, an oxygen atom, a sulphenyl, sulphinyl, sulphonyl, —NH, —NH—CO or —NH—CO—NH group and
Y₂ denotes a carbon-nitrogen bond or a carbonyl, sulphonyl, imino or —NH—CO group, while the carbonyl group of the —NH—CO group is linked to the nitrogen atom of the R₃NR₄ group, and the imino groups mentioned in the definition of the groups Y₁ and Y₂ may each additionally be substituted by a C₁₋₃-alkyl or carboxy-C₁₋₃-alkyl group,
a C₁₋₃-alkyl or C₃₋₅-cycloalkyl group substituted by a R₅NR₆ group, wherein
R₅ denotes a hydrogen atom, a C₁₋₃-alkyl, C₅₋₇-cycloalkyl, phenylcarbonyl, phenylsulphonyl or pyridinyl group and
R₆ denotes a C₁₋₃-alkyl, carboxy-C₁₋₃-alkyl or carboxy-C₁₋₃-alkylcarbonyl group,
a C₁₋₃-alkyl group which is substituted by a C₂₋₄-alkanoyl or C₅₋₇-cyclo-alkanoyl group and by a C₁₋₃-alkyl group substituted by a chlorine, bromine or iodine atom,
R_{b} denotes a hydrogen atom or a C₁₋₃-alkyl group and
R_{c} denotes a cyano group or an amidino group optionally substituted by one or two C₁₋₃-alkyl groups, wherein
the carboxy groups mentioned in the definition of the abovementioned groups may also be replaced by a group which may be converted in vivo into a carboxy group or by a group which is negatively charged under physiological conditions or
the amino and imino groups mentioned in the definition of the abovementioned groups may also be substituted by a group which can be cleaved in vivo, while
by a group which may be converted into a carboxy group in vivo is meant a hydroxymethyl group, a carboxy group esterified with an alcohol wherein the alcoholic moiety is a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula
R_{d}—CO—O—(RₑCR_{f})—OH,
wherein
R_{d} denotes a C₁₋₈-alkyl, C₅₋₇-cycloalkyl, phenyl or phenyl-C₁₋₃-alkyl group
Rₑ denotes a hydrogen atom, a C₁₋₃-alkyl, C₅₋₇-cycloalkyl or phenyl group and
R_{f} denotes a hydrogen atom or a C₁₋₃-alkyl group,
by a group which is negatively charged under physiological conditions is meant a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group
and by a group which can be cleaved from an imino or amino group in vivo is meant a hydroxy group, a benzoyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partly replaced by fluorine or chlorine atoms, a phenyl-C₁₋₆-alkoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, R_{d}—CO—O—(R_{d}CR_{f})—O—CO, C₁₋₆-alkyl-CO—NH—(R_{g}CRₕ)—O—CO or C₁₋₆-alkyl-CO—O—(R_{g}CRₕ)—(R_{g}CRₕ)—O—CO group, wherein R_{d} to R_{f} are as hereinbefore defined and
R_{g} and Rₕ, which may be identical or different, denote hydrogen atoms or C₁₋₃-alkyl groups,
the tautomers, stereoisomers, mixtures thereof and the salts thereof,
optionally in the form of the pharmaceutically acceptable acid addition salts thereof, as well as optionally in the form of the hydrates or solvates thereof, for preparing a pharmaceutical composition for the prevention or treatment of diseases from the group consisting of systemic inflammatory response syndrome (SIRS), sepsis and bacteraemia.

2. The method according to claim 1, wherein the benzimidazole used is (R)-2-(4-amidinophenylaminomethyl)-1-methyl-5-[1-(carboxymethylamino)-1-(pyrrolidinocarbonyl)-ethyl]-benzimidazole, optionally in the form of the pharmaceutically acceptable acid addition salts thereof, and optionally in the form of the hydrates or solvates thereof.

3. The method according to claim 2, wherein the monohydrochloride salt of (R)-2-(4-amidinophenylaminomethyl)-1-methyl-5-[1-(carboxymethylamino)-1-(pyrrolidinocarbonyl)-ethyl]-benzimidazole is used.

4. The method according to claim 1, wherein the condition is selected from the group consisting of SIRS caused by gram-positive pathogens, SIRS caused by gram-negative pathogens, SIRS caused by viruses, SIRS caused by single-cell eukaryotic parasites, SIRS caused by fungi, SIRS without organ failure, SIRS with organ failure, septic shock, septic syndrome, SIRS caused by pancreatitis, SIRS caused by systemic ischaemia, SIRS caused by organ-limited ischaemia, SIRS caused by trauma, SIRS occurring in connection with tumour diseases, SIRS caused by tissue damage, SIRS caused by burns, SIRS occurring after lengthy operations, SIRS as a consequence of organ transplants, SIRS as the result of shock of various kinds, SIRS caused by blood loss, SIRS as the result of cardiovascular failure, SIRS as the result of immuno-mediated organ failure, SIRS as the result of inflammatory reactions, SIRS as the result of treatment with inflammation mediators such as for example tumour necrosis factor alpha and/or beta and/or other cytokines, and also consisting of lung damage, acute lung injury and ARDS (acute respiratory distress syndrome), acute cardiovascular failure, organ failure after resuscitation, shock, kidney failure, cardiovascular failure, haematological damage, acidosis and multiple organ dysfunction syndrome (MODS) occurring in connection with SIRS.

5. The method of claim 1, wherein the pharmaceutical composition is intended as an accompanying treatment for bacteraemia.

6. The method of claim 1, wherein the pharmaceutical composition is intended for subcutaneous or parenteral and particularly intravenous administration.

7. The method of claim 1 further comprised of the step of co-administration of an inhibitor of platelet function.

8. The method of claim 7 wherein the inhibitor of platelet function is selected from the list consisting of acetylsalicylic acid, fibrinogen receptor antagonists, inhibitors of ADP-induced aggregation, P₂T receptor antagonists and combined thromboxane receptor antagonists/synthetase inhibitors.

9. The method of claim 1 further comprised of the step of co-administration of a thrombolytically active substance.

10. The method of claim 9 wherein the thrombolytically active substance is selected from the list consisting of alteplase, reteplase, tenecteplase, urokinase, staphylokinase and streptokinase.

11. The method of claim 1 further comprised of the step of co-administration of physiological activators and inhibitors of the clotting system and their recombinant analogues.

12. The method of claim 11 wherein the physiological activators and inhibitors of the clotting system are selected from the group consisting of Protein C, recombinant human activated Protein C, TFPI and antithrombin.

13. The method of claim 1 further comprised of the step of co-administration of substances with an antagonistic effect on endotoxins.

14. The method of claim 1 further comprised of the co-administration compounds selected from the list consisting of interleukins, TNF, bradykinin, prostaglandins, cyclooxygenases, NO, platelet activating factor, acetylhydrolases, inflammation inhibitors, immunosuppressant substances, antibiotics and catecholamines

15. Pharmaceutical composition comprised of
(a) at least one active substance selected from the group of benzimidazoles of general formula (I) 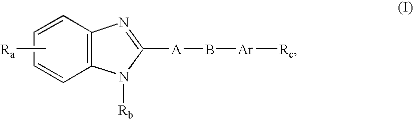
wherein
Ar denotes a phenylene or naphthylene group optionally substituted by a fluorine, chlorine or bromine atom, by a trifluoromethyl, C₁₋₃-alkyl or C₁₋₃-alkoxy group,
a thienylene, thiazolylene, pyridinylene, pyrimidinylene, pyrazinylene or pyridazinylene group optionally substituted in the carbon skeleton by a C₁₋₃-alkyl group,
A denotes a C₁₋₃-alkylene group,
B denotes an oxygen or sulphur atom, a methylene, carbonyl, sulphinyl or sulphonyl group, an imino group optionally substituted by a C₁₋₃-alkyl group wherein the alkyl moiety may be mono- or disubstituted by a carboxy group,
Rₐ denotes a R₁—CO—C₃₋₅-cycloalkyl group wherein
R₁ denotes a C₁₋₃-alkoxy, amino, C₁₋₄-alkylamino or di-(C₁₋₄-alkyl)-amino group, wherein in each case the alkyl moiety may be substituted by a carboxy group,
a 4- to 7-membered cycloalkyleneimino or cycloalkenyleneimino group which may be substituted by one or two C₁₋₃-alkyl groups, while an alkyl substituent may simultaneously be substituted by a hydroxy, C₁₋₃-alkoxy, carboxy, carboxy-C₁₋₃-alkoxy, carboxy-C₁₋₃-alkylamino, N—(C₁₋₃-alkyl)-N-(carboxy-C₁₋₃-alkyl)-amino, carboxy-C₁₋₃-alkylaminocarbonyl, N—(C₁₋₃-alkyl)-N-(carboxy-C₁₋₃-alkyl)-aminocarbonyl, carboxy-C₁₋₃-alkylaminocarbonylamino, 1-(C₁₋₃-alkyl)-3-(carboxy-C₁₋₃-alkyl)-aminocarbonylamino, 3-(C₁₋₃-alkyl)-3-(carboxy-C₁₋₃-alkyl)-aminocarbonylamino or 1,3-di-(C₁₋₃-alkyl)-3-(carboxy-C₁₋₃-alkyl)-aminocarbonylamino group,
a 4- to 7-membered cycloalkyleneimino group substituted by a hydroxy group,
a 5- to 7-membered cycloalkyleneimino group optionally substituted by a C₁₋₃-alkyl group, to which a phenyl ring is fused via two adjacent carbon atoms,
a morpholino, piperazino, N—(C₁₋₃-alkyl)-piperazino, pyrrolino, 3,4-dehydro-piperidino or pyrrol-1-yl group,
a R₂—CX—C₃₋₅-cycloalkyl group wherein
R₂ denotes a phenyl, naphthyl or monocyclic 5- or 6-membered heteroaryl group optionally substituted by a C₁₋₃-alkyl group, while the 6-membered heteroaryl group contains one, two or three nitrogen atoms and the 5-membered heteroaryl group contains an imino group optionally substituted by a C₁₋₃-alkyl group, an oxygen or sulphur atom or an imino group optionally substituted by a C₁₋₃-alkyl group and an oxygen or sulphur atom or one or two nitrogen atoms and the abovementioned alkyl substituent may be substituted by a carboxy, carboxy-C₁₋₃-alkoxy, carboxy-C₁₋₃-alkylamino or N—(C₁₋₃-alkyl)-carboxy-C₁₋₃-alkylamino group, and
X denotes an oxygen atom, a C₁₋₃-alkylimino, C₁₋₃-alkoxyimino, C₁₋₃-alkylhydrazino, di-(C₁₋₃-alkyl)-hydrazino, C₂₋₄-alkanoylhydrazino, N—(C₁₋₃-alkyl)-C₂₋₄-alkanoylhydrazino or C₁₋₃-alkylidene group each of which may be substituted by a carboxy group in the alkyl or alkanoyl moiety or in the alkyl and alkanoyl moiety,
a C₁₋₃-alkyl or C₃₋₅-cycloalkyl group substituted by an imidazole or imidazolone group, wherein
the imidazole ring may be substituted by a phenyl or carboxy group and by one or two C₁₋₃-alkyl groups or by one, two or three C₁₋₃-alkyl groups, while the substituents may be identical or different and one of the abovementioned alkyl substituents may simultaneously be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
the imidazolone ring may be substituted by a C₁₋₃-alkyl group, while the alkyl substituent may be substituted by a carboxy group or in the 2 or 3 position by an amino, C₂₋₄-alkanoylamino, C₁₋₃-alkylamino, N—(C₂₋₄-alkanoyl)-C₁₋₃-alkylamino or di-(C₁₋₃-alkyl)-amino group, and
additionally a phenyl or pyridine ring may be fused to the abovementioned imidazole and imidazolone rings via two adjacent carbon atoms,
an imidazolidin-2,4-dion-5-yl group which may be substituted by one or two C₁₋₃-alkyl groups, while simultaneously an alkyl substituent may be substituted by a carboxy group,
a C₁₋₄-alkyl group which is substituted
by a C₁₋₃-alkyl-Y₁—C₁₋₃-alkyl, HOOC—C₁₋₃-alkyl-Y₁—C₁₋₃-alkyl, tetrazolyl-C₁₋₃-alkyl-Y₂, R₃NR₄ or R₃NR₄—C₁₋₃-alkyl group and
by an isoxazolidinylcarbonyl group optionally substituted by a C₁₋₃-alkyl group, by a pyrrolinocarbonyl, 3,4-dehydro-piperidinocarbonyl, pyrrol-1-yl-carbonyl, carboxy, aminocarbonyl, C₁₋₃-alkylaminocarbonyl, di-(C₁₋₃-alkyl)-aminocarbonyl or 4- to 7-membered cycloalkyleneiminocarbonyl group, while in the abovementioned groups the cycloalkyleneimino moiety may be substituted by one or two C₁₋₃-alkyl groups and simultaneously in each case an alkyl moiety or alkyl substituent of the abovementioned C₁₋₃-alkylaminocarbonyl, di-(C₁₋₃-alkyl)-aminocarbonyl or cycloalkyleneiminocarbonyl groups may be substituted by a carboxy group, and the remaining hydrogen atoms of the C₁₋₄-alkyl group may be wholly or partly replaced by fluorine atoms, wherein
R₃ denotes a hydrogen atom or a C₁₋₃-alkyl group optionally substituted by a carboxy group and
R₄ denotes a hydrogen atom, a C₁₋₃-alkyl-Y₁—C₁₋₃-alkyl-Y₂, carboxy-C₁₋₃-alkyl-Y₁—C₁₋₃-alkyl-Y₂, C₁₋₃-alkyl-Y₂ or carboxy-C₁₋₃-alkyl-Y₂ group or
R₃ and R₄ together with the nitrogen atom between them denote a 4- to 7-membered cycloalkyleneimino group optionally substituted by a carboxy, C₁₋₃-alkyl or carboxy-C₁₋₃-alkyl group, wherein
Y₁ denotes a carbon-carbon bond, an oxygen atom, a sulphenyl, sulphinyl, sulphonyl, —NH, —NH—CO or —NH—CO—NH group and
Y₂ denotes a carbon-nitrogen bond or a carbonyl, sulphonyl, imino or —NH—CO group, while the carbonyl group of the —NH—CO group is linked to the nitrogen atom of the R₃NR₄ group, and the imino groups mentioned in the definition of the groups Y₁ and Y₂ may each additionally be substituted by a C₁₋₃-alkyl or carboxy-C₁₋₃-alkyl group,
a C₁₋₃-alkyl or C₃₋₅-cycloalkyl group substituted by a R₅NR₆ group, wherein
R₅ denotes a hydrogen atom, a C₁₋₃-alkyl, C₅₋₇-cycloalkyl, phenylcarbonyl, phenylsulphonyl or pyridinyl group and
R₆ denotes a C₁₋₃-alkyl, carboxy-C₁₋₃-alkyl or carboxy-C₁₋₃-alkylcarbonyl group,
a C₁₋₃-alkyl group which is substituted by a C₂₋₄-alkanoyl or C₅₋₇-cyclo-alkanoyl group and by a C₁₋₃-alkyl group substituted by a chlorine, bromine or iodine atom,
R_{b} denotes a hydrogen atom or a C₁₋₃-alkyl group and
R_{c} denotes a cyano group or an amidino group optionally substituted by one or two C₁₋₃-alkyl groups, wherein
the carboxy groups mentioned in the definition of the abovementioned groups may also be replaced by a group which may be converted in vivo into a carboxy group or by a group which is negatively charged under physiological conditions or
the amino and imino groups mentioned in the definition of the abovementioned groups may also be substituted by a group which can be cleaved in vivo, wherein
by a group which may be converted into a carboxy group in vivo is meant a hydroxymethyl group, a carboxy group esterified with an alcohol wherein the alcoholic moiety is a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol,
with the proviso that no bond to the oxygen atom starts from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula
R_{d}—CO—O—(RₑCR_{f})—OH,
wherein
R_{d} denotes a C₁₋₈-alkyl, C₅₋₇-cycloalkyl, phenyl or phenyl-C₁₋₃-alkyl group
Rₑ denotes a hydrogen atom, a C₁₋₃-alkyl, C₅₋₇-cycloalkyl or phenyl group and
R_{f} denotes a hydrogen atom or a C₁₋₃-alkyl group,
by a group which is negatively charged under physiological conditions is meant a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group
and by a group which can be cleaved from an imino or amino group in vivo is meant a hydroxy group, a benzoyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partly replaced by fluorine or chlorine atoms, a phenyl-C₁₋₆-alkoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, R_{d}—CO—O—(R_{d}CR_{f})—O—CO, C₁₋₆-alkyl-CO—NH—(R_{g}CRₕ)—O—CO or C₁₋₆-alkyl-CO—O—(R_{g}CRₕ)—(R_{g}CRₕ)—O—CO group, wherein R_{d} to R_{f} are as hereinbefore defined and
R_{g} and Rₕ, which may be identical or different, denote hydrogen atoms or C₁₋₃-alkyl groups,
the tautomers, stereoisomers, mixtures thereof and the salts thereof, and
(b) at least one active substance selected from the group consisting of inhibitors of platelet function such as in particular acetylsalicylic acid, fibrinogen receptor antagonists, inhibitors of ADP-induced aggregation, P₂T receptor antagonists and combined thromboxane receptor antagonists/synthetase inhibitors, thrombolytically active substances, such as in particular alteplase, reteplase, tenecteplase, urokinase, staphylokinase and streptokinase, physiological activators and inhibitors of the clotting system and their recombinant analogues such as in particular Protein C, recombinant human activated Protein C, TFPI and antithrombin, substances with an antagonistic effect on endotoxins, interleukins, TNF, bradykinin, prostaglandins, cyclooxygenases, NO, platelet activating factor acetylhydrolases, inflammation inhibitors, immunosuppressant substances, antibiotics and catecholamines

16. Pharmaceutical composition according to claim 15, comprised of
(a) (R)-2-(4-amidinophenylaminomethyl)-1-methyl-5-[1-(carboxymethylamino)-1-(pyrrolidinocarbonyl)-ethyl]-benzimidazole, optionally in the form of the pharmaceutically acceptable acid addition salts and optionally in the form of the hydrates or solvates thereof, and
(b) a PAF-AH or a PAF-AH derivative.

17. Pharmaceutical kit comprised of at least
(a) (R)-2-(4-amidinophenylaminomethyl)-1-methyl-5-[1-(carboxymethylamino)-1-(pyrrolidinocarbonyl)-ethyl]-benzimidazole, optionally in the form of the pharmaceutically acceptable acid addition salts and optionally in the form of the hydrates or solvates thereof, and
(b) a PAF-AH or a PAF-AH derivative.

18. Pharmaceutical kit comprised of least
(a) (R)-2-(4-amidinophenylaminomethyl)-1-methyl-5-[1-(carboxymethylamino)-1-(pyrrolidinocarbonyl)-ethyl]-benzimidazole, optionally in the form of the pharmaceutically acceptable acid addition salts and optionally in the form of the hydrates or solvates thereof, and
(b) a tumour necrosis factor alpha (TNF-alpha) antagonist.

19. Use of a PAF-AH or a PAF-AH derivative or a TNF-alpha antagonist for preparing a pharmaceutical composition for combined use with a (R)-2-(4-amidinophenylaminomethyl)-1-methyl-5-[1-(carboxymethylamino)-1-(pyrrolidinocarbonyl)-ethyl]-benzimidazole.
